(19) United States
(12) Patent Application Publication
(10) Pub. No.: US 2006/0269499 A1
(43) Pub. Date: Nov. 30, 2006

(54) Substantive anionic silicone oil emulsion compositions for hair and skin conditioning

(75) Inventors: John L. Gormley, Midland Park, NJ (US); Amit R. Shah, Commack, NY (US)

Correspondence Address: THE FIRM OF KARL F ROSS, 5676 RIVERDALE AVENUE, PO BOX 900, RIVERDALE (BRONX), NY, 10471-0900, US

(73) Assignee: GRANT INDUSTRIES, INC.

(21) Appl. No.: 11/440,814

(22) Filed: May 25, 2006

Related U.S. Application Data
(60) Provisional application No. 60/684,311, filed on May 25, 2005.

(51) Int. Cl. A61K 8/89

(52) U.S. Cl. 424/70.12

(57) ABSTRACT

Substantive silicone emulsions are disclosed containing a combination of polydimethylsiloxane and pyrrolidone carboxylic acid dimethylsiloxane copolymer. The emulsions are added to personal care compositions for the hair and skin to provide, smoothness, gloss and a conditioning feel to the user.

### CROSS REFERENCE TO RELATED APPLICATIONS

This application is related to copending Provisional Application 60/684,311 filed 25 May 2005.

### FIELD OF THE INVENTION

The present invention relates to substantive water dispersible silicone emulsions using a combination of polydimethylsiloxanes and pyrrolidone carboxylic acid dimethylsiloxane copolymers. The invention further relates to compositions for hair and skin treatment and to methods of treating the hair or skin.

### BACKGROUND OF THE INVENTION

Polydimethylsiloxanes are commonly used in conditioning and cleansing systems to provide smoothness, gloss and a conditioning feel characteristic of this material, especially when certain quaternary and fatty ingredients are included in the compositions to enhance deposition of silicone on hair or skin. They suffer from the drawbacks of not being water dispensible and frequently require homogenization and heat to incorporate into a formula.

U.S. Pat. No. 6,565,837 is the closest prior art and it discloses several other pyrrolidone carboxylic acid (PCA) composition of matter patents. In the patent there is disclosure of a solvate of PCA dimethicone in isopropyl alcohol (Example 6) and the treating of hair with conditioners and shampoos containing the solvated PCA dimethicone. These compositions are not emulsions. A disadvantage of these prior art compositions is that they require an alcohol solvent to work.

### OBJECT OF THE INVENTION

The object of the invention is to provide a water dispersible composition for treating the hair or the skin that yields a very high degree of beneficial conditioning properties and shine without the need to use an alcohol solvent.

### SUMMARY OF THE INVENTION

is The present invention is a water dispersible oil-in-water emulsion premix composition for personal care use comprised of:

a) about 60% to about 90% by weight of an oil phase containing a blend of about 5% to about 95% by weight polydimethylsiloxane fluid of Formula I 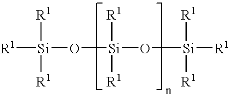
wherein R¹ is the same or different and is selected from C₁ to C₁₈ alkyl group or phenyl group, with the caveat that R¹ is at least 50% methyl groups and preferably all methyl groups, n is an integer from 10 to about 5000, preferably 500 to about 2500, more preferably around 1000 to 2000, about 5% to about 95% by weight of a pyrrolidone carboxylic acid dimethicone copolymer of Formula II 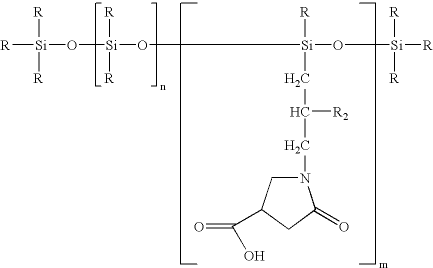
wherein:
R is selected from methyl or phenyl; R₂, is hydrogen or methyl, m is an integer from 1 to 20; and n is an integer from 20 to about 1000, and
about 0.5% to about 2% by weight of a low hydrophilic-lipophilic balance (HLB) emulsifier; and

b) about 10% to about 40% by weight of a water phase containing 0.5% to about 2% by weight of a high HLB emulsifier.

The polydimethylsiloxane and their derivative polymers included within Formula I are typically sold as fluids based on their viscosity, from 0.65 cst up to intractable gums and all have use in cosmetics. Low viscosity fluids spread well and may be volatile, but do not form a substantive film. High viscosity fluids can form a substantive film, but are not easily formulated in part because of resistance to spreading. Silicone fluid mixtures comprised of both low and high viscosity materials are frequently used to obtain a balanced property set. The viscosity of a fluid or mixture of fluids of Formula I according to this invention can be 50 to 1,000,000 centistokes (cSt.) preferably 10,000 to 100,000 cSt. and more preferably 50,000 to 100,000 cSt. These polymers are commercially available from Grant Industries as the Gransil DMF™ series (DMF=dimethyl fluids) and also from Dow Coming as the DC-200™ series. They may also be sold as emulsions, but only the neat polymers are applicable for the purposes of this invention.

About 5% to 95% by weight polydimethylsiloxane fluid of Formula I is blended with about 95% to 5% by weight of the pyrrolidone carboxylic acid dimethicone copolymer of Formula II, preferably 50% to 90% by weight of Formula I is blended with 10%-50% by weight of Formula II, more preferably about 60% to 80% by weight of Formula I is blended with about 20%-40% by weight of Formula II is used. The more preferred ratio will yield the best balance of silicone deposition and cosmetic properties as will be described in more detail.

The Formula II—pyrrolidone carboxylic acid dimethicone copolymers contain an acid group and can also be characterized according to a titration of acid number as typical of organic acids. Preferred acid number ranges from about 5 to about 25 milliequivalent acid/g. polymer.

The product Gransil PCA™ where R is methyl and R₂ is hydrogen, available from Grant Industries, is the preferred copolymer of the Formula (II). The mixed silicone polymers of Formulas I and II are the integral components of an oil phase that will become the internal phase in an oil-in-water emulsion. Other oil phase ingredients and surfactant are combined prior to emulsification. A minimum amount of a low HLB emulsifying surfactant is required to be added to the oil phase, typically about 0.2%-2% by weight of the finished emulsion. Typical low HLB surfactant are C₁₀ to C₁₈ alkyl alcohols reacted with about 2 to 6 equivalents of ethylene oxide. Laureth-2 and oleth-4 are typical examples of low HLB surfactants. McCutcheon's Vol. 1: Emulsifiers & Detergents, The Manufacturing Confectioner Publishing Co., Glen Rock, N.J. (1992) is a useful reference that illustrates many useful examples of low HLB surfactant that would work sufficiently well in this invention. For the present invention an emulsifier or surfactant with a low HLB number is one with a number below 15 and an emulsifier or surfactant with a high HLB number is one with a number of 15 or above. The oil phase can be 60-92% by weight of the final emulsion but 80%-90% oil phase is preferred. Optional oil phase components include, but are not limited to phenyl silicones, silicone elastomer gels, UV filters, vitamins, fatty esters, essential oils and dispersed inorganic or organic compounds like mica, pigments, nylon-12 or titanium dioxide. Total optional oil phase components are typically used at levels under 50% of the oil phase by weight, preferably under 25% by weight so as not to detract from the synergistic combination of non-ionic polydimethyl siloxane and the anionic pyrrolidone carboxylic acid containing polydimethylsiloxane in the emulsion.

The water phase comprises about 8 to 40% of the final emulsion weight and contains one or more high HLB surfactants at about 0.2% to about 2% by weight of the final emulsion. Useful high HLB surfactants include alkyl sulfates, alkyl ether sulfates, alkylpolyglucosides and ethoxylated alcohols, ethoxylated sugars and many other high HLB surfactants from McCutcheons Surfactant Guide. PLANTAREN 2000™ (decyl glucoside), available from Cognis, is an example of such a high HLB surfactant.

The compositions according to the invention include a combination of non-ionic and anionic silicone polymers that is made without heat or homogenization, is in emulsified form, is water dispersible and unexpectedly yields a very high degree of beneficial conditioning properties and shine. The emulsion is critical to the performance of the combined silicones, as water is the external phase and allows for the emulsion to be water dispersible and to form a lamellar structure that has slippery and glossy properties.

According to one feature of the present invention, the emulsion containing mixed dimethicone and a pyrrolidone carboxylic dimethicone copolymer is formulated into a personal care conditioning composition at 0.1 to about 20% by weight and at a final pH between about 3.5 to about 7.0 to provide unique hair and skin treatment properties. Preferred personal care compositions are leave-on lotions, rinse-off conditioners and cleansers for hair or body comprising the non-ionic/anionic emulsified silicone mixture compositions according to the present invention. Optionally, these compositions contain, quaternary agents, surfactants, vitamins, oils, fragrances, colors, drug actives, UV filters, thickening polymers and silicone acrylates. These and other embodiments will be described in more detail in the examples.

The emulsion according to the invention is prepared by slowly adding the oil phase to an adequately stirred water phase by employing a stirrer capable of handling high viscosity emulsions, particularly when high viscosity silicone fluids of Formula 1 are used. These and other attributes of the invention will be further delineated by the following examples:

### EXAMPLE 1 (COMPARATIVE)

An emulsion is prepared by the addition of an oil phase comprised of 78 g. Gransil PCA™ and 3 g. Brij 30 ™ (polyoxyethylated lauryl ether) to a stirring water phase comprised of 15 g. deionized water and 3 g. Plantaren 2000™ and 1% Germaben II™ preservative. The emulsion is thin with a viscosity under 2000 cst.

### EXAMPLE 2 (COMPARATIVE)

An emulsion is prepared by the addition of an oil phase comprised of 78 g. Gransil DMF-60,000™ and 3 g. Brij 30™ to a stirring water phase comprised of 15 g. water and 3 g. Plantaren 2000™ and 1% Germaben II™ preservative. The final emulsion is smooth with viscosity greater than 50,000 cSt.

### EXAMPLE 3 (PRESENT INVENTION)

An emulsion is prepared by the addition of an oil phase comprised of 58 g. 60,000 cSt polydimethylsiloxane fluid (Gransil DMF-60,000 by Grant Industries) and 20 g. Gransil PCA™ (a pyrrolidone carboxylic acid dimethicone copolymer by Grant Industries having an average molecular weight per carboxylic acid of about 4400 daltons) and 3 g. Brij 30™ (Uniqema) to a stirring water phase comprised of 15 g. water and 3 g. Plantaren 2000™ (from Cognis) and 1% Germaben II™ preservative (from International Specialty Products). The final emulsion is smooth with a viscosity of about 10,000 cst.

### EXAMPLE 4

### Leave-On and Rinse Off Conditioner Tests for the Emulsion

In three cycles of testing, 1 gram of emulsion from Examples 1 to 3 was applied to the hands of eight panelists and the performance attributes collected for leave-on conditioning feel, rinse-off conditioning feel and dry after-feel. Example 1 emulsion was reported to be sticky for all test parameters. Example 2 emulsion was reported to feel good initially, but completely rinsed away, leaving little, if any after feel. Example 3 emulsion (present invention) was unanimously reported to have significantly higher initial conditioning feel, lasting wet slip, high gloss and a long lasting and only slightly tacky after-feel. This result shows it is the combination of the polydimethyl siloxane with the pyrrolidone carboxylic acid siloxane in emulsified form synergistically gives the best conditioning effect. The examples were prepared by separately mixing Part A and Part B ingredients at 65° C., followed by combining Part B to Part A slowly with mixing. Next cool to 45° C. and add part C with continued stirring until cooled to room temperature.

### EXAMPLES 5 AND 6

### Rinse-Off Conditioner

| | | Ex. 5 | Ex. 6 |
|---|---|---|---|
| | | (Inventive) | (Comparative) |
| PART | Ingredient | % by weight | % by weight |
| A | Cetyl Alcohol | 4.00 | 4.00 |
| | Carbowax 8000 | 3.00 | 3.00 |
| | (Noveon) | | |
| | Incroquat Behenyl | 2.40 | 2.40 |
| | TMS (Croda) | | |
| | Synthetic | 1.00 | 1.00 |
| | Spermacetti Wax | | |
| | Lanolin | 0.70 | 0.70 |
| | Abil EM-90 | 1.00 | 1.00 |
| | (Degussa) | | |
| B | DI H20 | 82.00 | 82.00 |
| | Hydroxyethylcellulose | 0.40 | 0.40 |
| | Germaben-II | 0.50 | 0.50 |
| C | Example 3 emulsion | 5.00 | — |
| | Example 2 emulsion | — | 5.00 |
| | Citric acid adjustment | to pH 5 | to pH 5 |
| Performance | | Excellent | Fair |

Each product was tested on four panelists who ranked manageability, shine and conditioning of each product on hair. Example 5 with the inventive emulsion system was excellent and the comparative Example 6 was ranked fair. This example illustrates how the inventive emulsion can be post added to an oil in water emulsion.

The emulsion of Example 3 was formulated in conditioning shampoos

### EXAMPLE 7

| Ingredients | Phase | Amount(g) |
|---|---|---|
| Water | A | 19.98 |
| WAQ ALS (Cognis) | A | 37.93 |
| Standapol ES-40 (Cognis) | A | 15 |
| 3% Polyquaternium-10 in water | A | 11.67 |
| 29% Arquad 16-29 Cetrimonium CI (Akzo) | A | 1.72 |
| Cocamide MIPA | B | 3 |
| Behenyl alcohol | B | 2 |
| Ethylene Glycol distearate | B | 1.5 |
| Tricetylmonium CI | B | 0.5 |
| Product from Example 3 | C | 5 |
| 50% Citric Acid | C | 0.2 |
| Germaben II | C | 0.5 |
| Ammonium CI | C | 1 |
| Total | | 100 |

The emulsion of Example 3 was formulated into a conditioning shampoo. The phase A ingredients were combined and mixed and heated to 65° C. Separately combine phase B ingredients, heat to 65° C. and add slowly to stirring water phase with good agitation. Remove heat, add Phase C when under 45° C., continue mixing until cool.

### EXAMPLE 8 (PRESENT INVENTION)

An emulsion is prepared by the addition of an oil phase comprise of 62 g 60,000 cSt polydimethylsiloxane fluid and 20 g GransilPCA, 0.075 g NaOH, 0.5 g. Simulgel EG (Seppic) and 1 g BRIJ 3D (Uniquema) to a stirring vessel phase comprised of 10 g of water, 5 g of 1,3-butylene glycol and 1 g Plantaren 2000 (from Cognis) and 1% Germaben II preservative (from International Specialty Products). The final emulsion is smooth with a viscosity of about 50,000 cSt.

### EXAMPLE 9 (PRESENT INVENTION)

An emulsion is prepared by the addition of an oil phase comprised of 62 g 60,000 cSt polydimethylsiloxane fluid, 30 g Granacrylsil BMAS (a silicone acrylate copolymer in isododecane by Grant Industries Inc. and 20 g Gransil PCA, 0.075 g NaOH, and 1 g Brij 30 (Uniquema) to a stirring water phase comprised of 10 g of water, 5 g of 1,3-butylene-glycol and 1 g Plantaren 2000 (from Cognis) and 1% Germaben II preservative (from International Specialty Products). The final emulsion is smooth with a viscosity of about 50,000 cSt.

What is claimed is:

1. An oil-in-water emulsion composition which comprises:
(a) about 60% to about 90% by weight of an oil phase containing a blend of about 5% to about 95% by weight polydimethylsiloxane fluid of Formula I 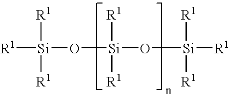
wherein each R¹ is the same or different and is selected from C₁ to C₁₈ alkyl group or phenyl group, with the caveat that R¹ is at least 50% methyl groups, n is an integer from about 10 to about 5000, about 5% to about 95% by weight of a pyrrolidone carboxylic acid dimethicone copolymer of Formula II 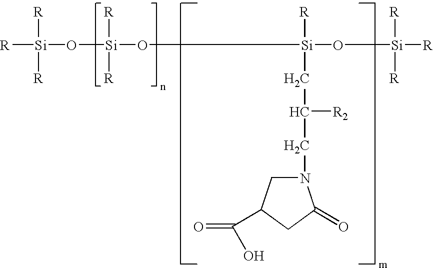
wherein:
R is selected from methyl or phenyl; R₂, is hydrogen or methyl, m is an integer from 1 to about 20; and n is an integer from about 20 to about 1000, and about 0.5% to about 2% by weight of a low HLB emulsifier; and
(b) about 10% to about 40% by weight of a water phase containing 0.5% to about 2% by weight of a high HLB emulsifier.

2. The oil-in-water emulsion composition defined in claim 1 wherein in the polydimethylsiloxane fluid of Formula I each of the groups R¹ is a methyl group.

3. The oil-in-water emulsion composition defined in claim 1 wherein in the polydimethylsiloxane fluid of Formula I n is an integer of about 500 to about 2500.

4. The oil-in-water emulsion composition defined in claim 1 wherein in the polydimethylsiloxane fluid of Formula I n is an integer of about 1000 to about 2000.

5. The oil-in-water emulsion composition defined in claim 1 wherein the low HLB emulsifier is a C₁₀ to C₁₈ alkyl alcohol reacted with about 2 to 6 equivalents of ethylene oxide.

6. The oil-in-water emulsion composition defined in claim 5 wherein the low HLB emulsifier is laureth-2, oleth-4, or a polyoxyethylated lauryl ether.

7. The oil-in-water emulsion composition defined in claim 1 wherein in the pyrrolidone carboxylic acid dimethicone copolymer of Formula II, R is methyl and R₂ is hydrogen.

8. The oil-in-water emulsion composition defined in claim 1 wherein the high HLB emulsifier is a water-soluble alkylpolyglucoside, ethoxylated alcohol, or ethoxylated sugar.

9. The oil-in-water emulsion composition defined in claim 8 wherein the water-soluble ethoxylated sugar is glucose or sorbitol.

10. The oil-in-water emulsion composition defined in claim 1 wherein the high HLB emulsifier is decyl glucoside.

11. A personal care composition which comprises:
(i) about 0.1 to about 20% by weight of the oil-in-water emulsion composition defined in claim 1; and
(ii) a cosmetically effective amount of a lotion, conditioner or cleanser for hair or body, wherein the pH of the composition is about 3.5 to about 7.0.

12. The personal care composition defined in claim 11 in the form of a rinse-off conditioner.

13. The personal care composition defined in claim 11 wherein the rinse-off conditioner or cleanser for the hair is a conditioning shampoo.

14. A method of preparing an oil-in-water composition which comprises
(a) about 60% to about 90% by weight of an oil phase containing a blend of about 5% to about 95% by weight polydimethylsiloxane fluid of Formula I 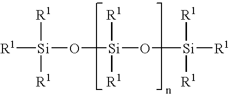
wherein each R¹ is the same or different and is selected from C₁ to C₁₈ alkyl group or phenyl group, with the caveat that R¹ is at least 50% methyl groups, n is an integer from about 10 to about 5000, about 5% to about 95% by weight of a pyrrolidone carboxylic acid dimethicone copolymer of Formula II 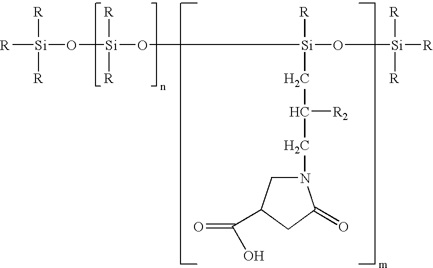
wherein:
R is selected from methyl or phenyl; R₂, is hydrogen or methyl, m is an integer from 1 to about 20; and n is an integer from about 20 to about 1000, and about 0.5% to about 2% by weight of a low HLB emulsifier; and
(b) about 10% to about 40% by weight of a water phase containing 0.5% to about 2% by weight of a high HLB emulsifier, comprising the step of
slowly adding the oil phase to the water phase under stirring by employing a stirrer capable of handling a high viscosity emulsion to obtain a smooth emulsion composition with a viscosity of about 10,000 cps.

15. A method of preparing the personal care composition defined in claim 11 which comprises the steps of
(a) preparing the leave-on lotion, rinse-off conditioner or cleanser for hair or body at a temperature of about 65° C.;
(b) cooling the leave-on lotion, rinse-off conditioner or cleanser for hair or body obtained during step (a) to a temperature of about 45° C.; and
(c) adding the oil-in-water emulsion while maintaining the pH of the personal care composition between 3.5 and 7.
